(19) United States
(12) Patent Application Publication
(10) Pub. No.: US 2015/0111778 A1
(43) Pub. Date: Apr. 23, 2015

(54) BIO-NANO-CHIP FOR ANTICONVULSANT DRUG SALIVARY ASSAY

(71) Applicants: WILLIAM MARSH RICE UNIVERSITY, Houston, TX (US); BOARD OF REGENTS OF THE UNIVERSITY OF TEXAS SYSTEM, Austin, TX (US)

(72) Inventors: John T. MCDEVITT, Houston, TX (US); Nicolaos CHRISTODOULIDES, Houston, TX (US); Giridhar P. KALAMANGALAM, Houston, TX (US)

(21) Appl. No.: 14/503,400

(22) Filed: Oct. 1, 2014

Related U.S. Application Data
(60) Provisional application No. 61/893,588, filed on Oct. 21, 2013.

(51) Int. Cl. G01N 33/94; G01N 33/543; B01L 3/00; G01N 33/545

(52) U.S. Cl. CPC G01N 33/9473; G01N 33/545; G01N 33/54366; B01L 3/502761; G01N 33/54306; B01L 2300/0636; B01L 2300/0867; B01L 2300/087; B01L 2200/0668; B01L 2300/0819; G01N 2800/2857
USPC 506/9; 506/15; 506/18

(57) ABSTRACT

This disclosure describes portable bio-nano-chip (BNC) assays of antiepileptic drugs (AED) using salivary samples. The BNC technology results in a more convenient, less expensive, and less time consuming sampling and analysis.

### PRIOR RELATED APPLICATIONS

This application claims priority to U.S. Provisional Application No. 61/893,588, filed Oct. 21, 2013, which is incorporated by reference in its entirety herein for all purposes.

### FEDERALLY SPONSORED RESEARCH STATEMENT

Not applicable.

### FIELD OF THE DISCLOSURE

The disclosure generally relates to Bio-nano-chip (BNC) technology and specifically to antiepileptic drugs (AED)-specific saliva assay using BNC.

### BACKGROUND OF THE DISCLOSURE

Epilepsy is a common disabling neurological condition affecting three million

Americans and 50 million persons worldwide, typically requiring drugs such as those shown in FIG. 3 to control. Blood tests in patients taking antiepileptic drugs (AEDs) may have several purposes: monitoring compliance, following the results of AED dosage changes, establishing a patient's maximum tolerated serum level, and looking for early signs of adverse effects (e.g., hepatic, renal, hematopoietic). The anti-seizure effects of AEDs are optimal when steady therapeutic concentrations are achieved; drops in body drug levels can quickly lead to breakthrough seizures. Regular drug level assay therefore is crucial for managing epilepsy.

Serum drug monitoring is a routine practice that is vital to patient care. The current method of drug monitoring has been in existence for over 50 years—blood draw, followed by assay of the analyte of interest (the drug itself or a metabolite of the drug) in a clinical chemistry laboratory. This method, though well-established, is inefficient, often inconvenient, impractical in certain patient populations, expensive, and demanding of specialized facilities and personnel. Furthermore, this method is invasive and sampling may be difficult to obtain just before or during epileptic episodes.

As such, there has been much research in using oral fluid in lieu of serum. Oral fluid has been found to have AED concentrations that strongly correlate with serum concentrations. Furthermore, saliva collection is advantageous because it is painless and noninvasive, and untrained personnel can easily be taught and complete the collection process. Remote patients could mail saliva samples to a laboratory for monitoring, and samples could be obtained in the immediate postictal state to provide a “real-time” concentration.

The McDevitt Research Group of Rice University (formerly of the University of Texas at Austin) has been developing and perfecting a novel bead based assay system called Programmable Bio-Nano-Chip and described in e.g. WO2012154306, WO2012065117, and WO2012065025.

Programmable Bio-Nano-Chip (pBNC) utilizes microfluidics and advanced biochemistry to provide a rapid and easy-to-use method for obtaining quantitative biomarker assessments with potential use at the point-of-care. By utilizing the principles of microfluidics and a lab-on-a-chip approach, the pBNC assays provide a way for monitoring multiple biomarkers simultaneously, require drastically reduced volumes of chemical reagents, and can provide a biomarker diagnosis in minutes as compared to the week long-wait times of market available lab-based tests.

The bio-specimens and reagents are guided and delivered via a set of microfluidic pathways, etched into the assay, onto the microbeads where the reaction takes place. Once the sample and reagents have arrived at the microbeads, a set of biochemical reactions take place which trigger the beads to fluoresce proportionally to the concentration of the biomarker of interest. Digital images of these beads can then be obtained using a simple laboratory-based fluorescence microscope or optical device and then passed through custom built image processing software to convert the fluorescent intensity into a biomarker concentration.

McDevitt et al. programmable-bio-nano-chip platform of 61/498,761 and US20120322682, for example, has been used to perform live and fixed cellular imaging on a microfluidic scale that helps to reduce reagent consumption, and improve transport of fluorescent reagents to 3D-culture models to serve as an integrated imaging platform.

There exist a need in the art for a simple and quick method for assaying drugs used to treat epilepsy. Ideally, a person with epilepsy or caregiver could monitor and track the concentration of anti-epilepsy drug concentrations at home as well as during medical treatment.

### SUMMARY OF THE DISCLOSURE

This disclosure relates to a bio-nano-chip (BNC)-based assay for detecting the concentration of anti-epileptic drug concentrations in a person's saliva. Such a chip can be used with the laboratory based p-BNC instrumentation, the portable BNC assay system or a hand held device designed for home use that is akin to blood sugar self-monitoring by diabetics.

The BNC is a packaged microfluidic sample processing and immune-analysis chip that serves as the functional component for the detection and quantitation of the drug and metabolites.

Typically, a reverse competitive type of immunoassay is run with the BNC because it is easier to bind the fluorophore to the antibody than to the drug. Plus, the assay needs only a single labeled antibody per drug and the tests are more sensitive or can detect lower levels of drugs when set up this way. The array included beads coupled to BSA-drug conjugates (drug sensors), beads coated with BSA alone (that serve as negative controls and indicators of the specificity of the reactions that take place within the card), as well as beads loaded with fixed amounts of photo-stable fluorophore that serve as calibrators of the LOC system. The array also offers a bead redundancy that contributes to higher accuracy and precision of LOC drug measurements. In the absence of drug in the sample, the tracer antibody (a labeled antibody) specifically bound the drug sensors and produced a strong signal on the surface as well as within the interior of the porous bead. In the presence of drug in the sample, the binding of the tracer was reduced in a drug-specific, dose-dependent manner. Use of drug standards allowed for the generation of dose response curves, which were then used to interpolate the concentration of the drug in unknown samples.

In the current invention, the BNC has a bead-array platform with beads coated with BSA-drug conjugate, negative controls beads coated with BSA alone, and calibrator beads used as internal controls. This bead-based test uses non-invasive oral fluid sampling. Such a sampling involves use of an oral swab to brush the entire upper and lower gum line or collection of drool with a pipette. If a swab is used, then extraction of the saliva involves insertion of the swab into a specimen collection tube with the assay fluid that includes the tracer antibody used in this particular competitive type of an immunoassay. Alternatively, if the oral fluid is collected by pipette, then it can be diluted directly into the assay fluid. The sample/tracer mixture is then delivered to the p-BNC flow cell equipped with a bead-array platform.

Preferably, the cartridge also has internal microfluidics on said substrate for carrying fluid to and from said bead sensors, and a sample entry port. In some embodiments, the drug testing cartridge can include at least one reagent blister fluidly connected to said bead sensors, and/or at least one waste fluid chamber fluidly connected to and downstream of said bead sensors. However, in other embodiments, these can be provided by the analyzer.

In the absence of the targeted drug in the sample, the tracer antibody specifically recognizes its corresponding drug sensor(s), producing a strong signal on the surface, as well as within the interior of the porous bead in the array. In the presence of the targeted drug in the sample the binding of the tracer is reduced in a drug specific, dose dependent manner. The array allows for 3-4 bead redundancy of bead sensors per drug target, which translates into higher accuracy and more precise measurements.

The novel aspect of the present disclosure is that it utilizes saliva monitoring of AED drugs, wherein the saliva sampling can be taken anywhere using a swab. This would provide information regarding AED drug concentrations during epileptic episodes or shortly thereafter.

Another novel aspect is that the presently disclosed assay integrates the various p-BNC assay systems allowing for laboratory testing, point-of-care testing, and hand-held testing of the sample. This would give a person the option of taking a sample at various times, such as during the postictal state after a seizure, and mailing to a testing center or running the sample at home. For at home use, the present disclosure would allow for monitoring and changing medication dosage to prevent hazardous dips in AED concentration. This is akin to the at home blood-sugar testing that people with diabetes perform. Thus, a person suffering from epilepsy could e.g. take a dose early if the drug concentration becomes low enough to potentially trigger a seizure.

One embodiment of the present system is a customized pBNC card for testing AED concentration. The pBNC has one or more beads coated with BSA-drug conjugate, negative controls beads coated with BSA alone, and calibrator beads used as internal controls. The pBNC can be customized such that the targeted drugs are those being prescribed to a patient. For instance, a pBNC card testing for phenobarbital concentrations only would be used by a person taking this AED, whereas another pBNC might have beads for targeting phenobarbital and phenytoin for a person taking both AEDs.

The invention can also be a home-kit comprising the BNC as described above, wrapped in an airtight package, together with a vial of assay buffer, a swab or other sample collection device and a syringe for transferring sample to BNC, instructions and the like and a home analyzer. A person could obtain a sample swabbing the gums and cheeks or by collecting drool and place the sample into the vial of assay buffer. The assay buffer/sample could then be injected into the BNC, which would be placed inside the analyzer during the immunoassay.

The pBNC can be of various sizes so as to fit the cell dimensions for the laboratory assay system, the portable system, or the hand-held device.

The terms “oral fluid” and “saliva” are interchangeable and refers to the clear, tasteless, odorless, slightly acid (pH 6.8) fluid, consisting of the secretions from the parotid, sublingual, and submandibular salivary glands and the mucous glands of the oral cavity.

By “reader” or “detector” or “analyzer” what is meant is a device that contains the optics, optic sensing means, processor, user interface, and fluidics and is the device that runs the assays described herein and thus “analyzes” the sample and “reads” or “detects” the results.

By “card” or “cartridge” what is meant is a generally planar substrate having microfluidic channels and chambers therein, as well as one or more access ports, and houses the bead array specific for the drug testing assays described herein.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| pBNC | Programmable Bio-nano-chip |
| AED | Anti-epileptic drug |
| PBT | Phenobarbital |
| PHY | Phenytoin |
| BSA | Bovine Serum Albumin |

### BRIEF DESCRIPTION OF THE DRAWINGS

FIG. 1 shows the laboratory-based p-BNC instrumentation for reading the assays and the commercial, portable unit. (A) Laboratory based station and flow cell. This platform consists of distinct units, such as a personal computer (i) that controls all functionalities, including fluid delivery, image capture and analyses, (ii) a customized fluorescent microscope equipped with a video camera (i.e. charged-coupled device). In this system, the bead-loaded chip is sandwiched between two optically transparent polymethylmethacrylate (PMMA) inserts, packaged within a metal casing described as the ‘flow cell’ (iii). A fluid delivery system (iv) allows for delivery of sample and reagents to the microchip and the associated beads. B) The in development portable p-BNC analyzer (v) and single-use micro assay lab card (vi).

FIG. 2 displays an exemplary piping diagram for the pBNC microfluidic network.

FIG. 3 displays exemplary anti-epileptic drugs. A) Valproic acid, B) Phenobarbital, C) Phenytoin, D) clonazepam, E) carbamazepine, and F) Oxcarbazepine.

FIG. 4 shows a schematic of the oral-fluid based sampling procedure according to the present disclosure. Programmable BNC/oral fluid-based drug tests. Oral fluid sample collected by a swab (Ai) is extracted in assay buffer (Aii) and then delivered to the microfluidic cell hosting bead sensors arrayed on a microchip. In (B) shown are the schematic decoding the immuno-components of this competitive assay approach. In (C) shown are charge-coupled device (CCD)-captured images of a concentration-dependent response for the competitive-type p-BNC-based drug test (i—control 0, ii-10 and iii-100 ng/mL drug). Noted are a) the decrease in signal acquired on the drug sensors (D sensor) in response to the drug, b) the absence of signal on negative control beads (-ye CTL) and c) the consistency in signal intensity on the calibrator beads upon completion of three independent assay runs.

FIGS. 5A-C shows proof of concept for testing concentrations of phenytoin. FIGS. 5D-E shows the dose response for phenobarbital.

FIG. 6 displays the bead array setup in the BNC used in Example 1.

FIG. 7 displays raw images of BNC response to phenobarbital and phenytoin at different concentrations under a 300 ms exposure and 100% bulb output.

FIG. 8A-B display dose response curves and limits of detection for A) Phenytoin and B) Phenobarbital.

FIG. 9 displays clinical sample results with duplex assay for phenytoin and phenobarbital and the assay's agreement with a gold standard assay (Serum). Grey boxes show those values used for the comparison that are close to agreement.

FIG. 10 shows various bead image analysis methods: Line profile (LP), circular area of interest (cAOI), integrated density (ID), circular profile (CP) and fixed AOI, for the generation of dose response curves for NBC-based assay for THC. Each bead from the array was probed with these different data analysis strategies. Dose response curves generated by each method were compared and the method, or combination of methods, that provided the most sensitive and wide detection capabilities were selected as the optimal image analysis approach for subsequent experiments. Line profile and circular area of interest were the most informative data analysis method, and exemplary data for the LP analysis is shown in graphic form on the bottom. 422 ms is the exposure time of the CCD (camera) of the optical sensor. The dotted line on the left graph is the threshold signal intensity that defines the LOD for this assay. The threshold signal intensity (179.58) is defined by the signal intensity of the zero drug condition (i.e. +0 CONTROL) minus 3X times the standard deviation of the same zero drug standard run. The threshold signal intensity is then applied on the graph on the right. It is plotted as SI (large dot) and interpolated from the graph on the right to provide the LOD of 4.95 ng/mL for this assay (X-axis, see dashed line). The other dotted lines are results from the testing of unknown samples.

### DETAILED DESCRIPTION

The invention includes any of the following embodiments, in any combination:

A disposable drug testing cartridge comprising a generally flat substrate having thereon individual bead sensors arranged in an array, wherein each bead sensor is a porous polymeric bead having a drug bound thereto, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, oxcarbazepine, and biological metabolites or derivatives of same.
A disposable drug testing cartridge further comprising internal microfluidics on said substrate for carrying fluid to and from said bead sensors.
A disposable drug testing cartridge further comprising a sample entry port.
A disposable drug testing cartridge further comprising at least one reagent blister fluidly connected to said bead sensors.
A disposable drug testing cartridge further comprising at least one waste fluid chamber fluidly connected to and downstream of said bead sensors.
A disposable drug testing cartridge further comprising positive and negative control bead sensors and calibrator bead sensors having known amounts of a drug being calibrated.
A disposable drug testing cartridge wherein every drug bead sensor is present in said array in at least duplicate.
A disposable drug testing cartridge, wherein every drug bead sensor is present in said array in at least triplicate.
A disposable drug testing cartridge wherein said drug is conjugated to said bead sensor via a linker.
A disposable drug testing cartridge further comprising one or more of the following:

one or more reagent chambers fluidly connected to and upstream of said array;

one or more waste fluid chambers fluidly connected to and downstream of said array; a sample inlet upstream and fluidly connected to said one or more reagent chambers; and wherein each bead sensor is a porous polymeric bead of size between 50-300 nm±10%.

An assay for the monitoring of anti-epilepsy drug concentration in saliva, said assay comprising:

a) obtaining a sample of oral fluid from a patient; and

b) immunologically testing said sample to determine the level of anti-epileptic drugs;

c) wherein said testing is conducted on an array of agarose beads, conjugated to anti-epileptic drugs, and wherein signal from said array of agarose beads is analyzed by circular area of interest or line profiling or both.

A assay of monitoring anti-epilepsy drug concentration in saliva, wherein said anti-epileptic drugs are selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites of same.
An anti-epileptic drug testing assay system comprising:

a) a microfluidic lab-on-chip based reverse competitive immunoassay that comprises a disposable cartridge and a separate reader, wherein said cartridge fits into a slot on said reader, and said reader performs said competitive immunoassay and outputs a result;

b) said cartridge comprising:
i) a generally flat substrate having embedded microfluidic channels connecting an inlet port to an embedded downstream assay chamber having a transparent cover and containing a removable array of bead sensors;
ii) one or more reagent chambers fluidly connected to and upstream of said assay chamber; and
iii) one or more waste fluid chambers fluidly connected to and downstream of said assay chamber;
iv) wherein each bead sensor is a porous polymeric bead of size between 50-300 nm±10% having a drug conjugated thereto, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites of same; and

c) wherein said reverse competitive immunoassay has a lower limit of detection for each of said drugs of <50 ng/ml and a detection range of at least four orders of magnitude.

A drug testing assay, said cartridge comprising 4 or more of said drugs.
A drug testing assay, said cartridge comprising each of said drugs.
A kit, comprising the cartridge herein described wrapped in an airtight package, a vial of assay fluid, and an oral swab. The kit can include other components, e.g., instructions for use.
An anti-epileptic drug home testing assay kit comprising:

a) a microfluidic lab-on-chip based reverse competitive immunoassay that comprises a disposable cartridge;

b) a sample collection and solubilization device comprising:
i) a container closed with a removable cap;
ii) a lower portion of said container having an assay fluid separated from an upper portion of said container by a piercable membrane;
iii) said cap comprising a flexible bulb passing through said cap and fluidly connected to a hollow stem ending in a point, a lower portion of said stem being coated with an absorbent or bristled material for collecting a biological sample;
iv) wherein said device is proportioned to store said cap with said stem and said swab in said upper portion of said container, but can reach said buffer when said point pierces said piercable membrane, and said flexible bulb can be used to draw up and deliver said buffer.

c) said cartridge comprising:
i) a generally flat substrate having embedded microfluidic channels connecting an inlet port to an embedded downstream assay chamber having a transparent cover and containing a removable array of bead sensors;
ii) one or more reagent chambers fluidly connected to and upstream of said assay chamber;
iii) one or more waste fluid chambers fluidly connected to and downstream of said assay chamber; and
iv) wherein each bead sensor is a porous polymeric bead of size between 50-300 nm±10% having a drug conjugated thereto, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites of same.

The disclosure provides a biomarker assay cards for detecting anti-epileptic drugs in saliva and methods of analyzing the assay cards. Specifically, Bio-Nano-Chip (pBNC) biomarker assay cards are used to detect the concentration of AEDs using non-invasive sampling of saliva. These cards can be ‘read’ using a lab-based system, a portable system, or a hand held device for home use.

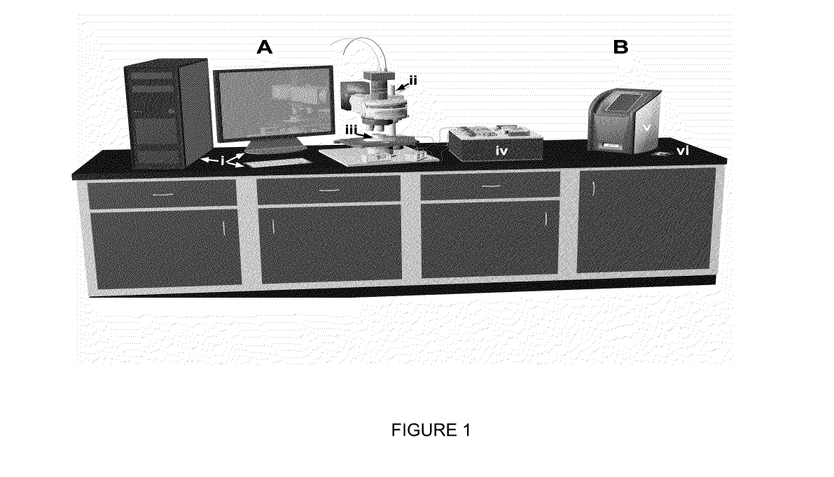

The lab-based system and a portable BNC reader system are seen in FIG. 1. Both systems have a customized fluorescent microscopy equipped with a charged coupled device (CCD), a fluid delivery system, a computer and a stage for insertion of the BNC cell. Generally, fluid is delivered in a microfluidic structure (BNC chip), flowing over and around the bead array, exiting underneath each microsphere through square fluid outlets. Using this setup, known or unknown concentrations of analytes of interest are delivered to the bead array where they bind to the primary antibodies and are captured in a second step using fluorophore pre-labeled secondary antibodies. The fluorescent signal is measured using a fluorescent microscope.

Not shown is the hand held device. A hand held device is for home use to identify potentially hazardous dips in AED concentrations, much like a diabetic monitors blood sugar level. The hand held device will have the CCD and a micro-computer for displaying results, as well a temperature control if needed, fluidics for moving fluid, possibly reagents or reagent inputs.

The CCD camera is only one option for assessing the light produced in the assay, and other photodetectors could be used including the common camera phone, CMOS sensor, photo diode, and the like. Further, the functionality can be provided in two components, e.g., the hand held unit containing fluidics and/or pump, temperature control (if needed), and a separate camera phone with dedicated software application for reading and displaying results.

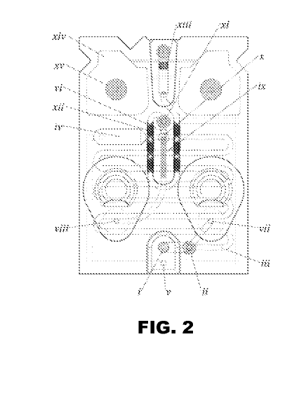

The reader devices will be capable of analyzing the BNC assays for AED. FIG. 2 displays the piping diagram of an exemplary pBNC chip like the ones used in the present disclosure. Other footprints are also suitable and available.

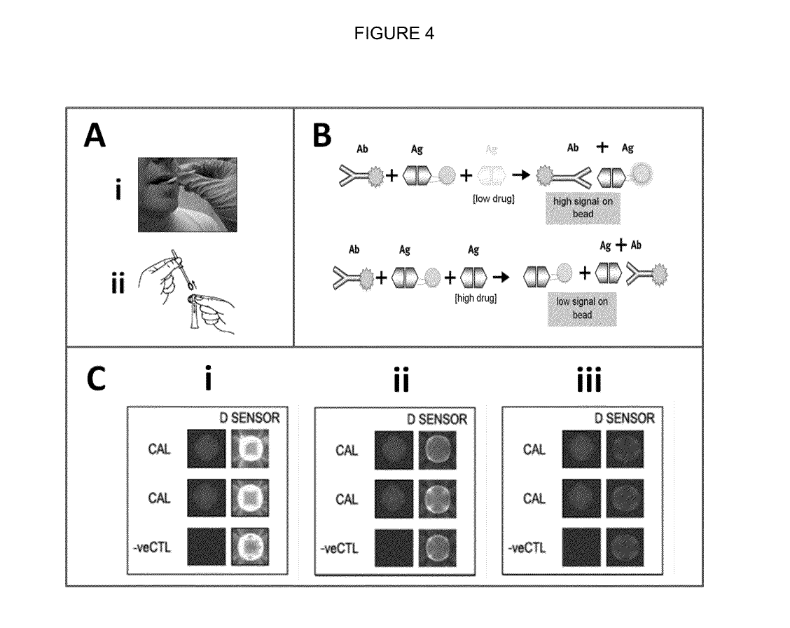

The present invention is exemplified with respect to FIGS. 4-10. However, these figures are exemplary only, and the invention can be broadly applied to any AED capable of being monitored in saliva. The following examples are intended to be illustrative only, and not unduly limit the scope of the appended claims.

In more detail, the BNC is a cartridge (see e.g., FIG. 2) comprising a substrate having inlets and microfluidics for moving fluid and a plurality of individual bead sensors wherein each bead sensor is a porous polymeric bead having a competitor drug bound thereto (either covalently bound or just absorbed, adsorbed, or adhered thereto). Additional beads for calibration and positive or negative control are also present. Here the piping diagram shows the principle microfluidic channels and features of the p-BNC's microfluidic network. The sample metering channel features (i) a port for sample input configured with a membrane filter, (ii) bubble trap, (iii) 100 μL capacity metered sample loop and (iv) a 20 μL sample overflow chamber leading to (v) an external vent. A passive pressure barrier (vi) prevents flow outside of the sample loop during loading. The right-hand fluid input port (vii) intersects the sample loop at a strategic location to evacuate the metered portion of sample toward the bead sensors. The lefthand fluid input hole (viii) is connected to the solid-state reagent storage chamber (ix) followed by an inline track-etch membrane filter (x) and bubble trap (xi). Both the sample metering channel and reagent preparation channel confluence at the distal bubble trap, which forms a junction column with a single output. The common channel features six staggered herringbone mixer sets (xii) leading to the bead sensors (xiii) and a large capacity bilateral waste (xiv). The reservoir vents (xv) are covered with selectively permeable vent membranes for secure waste containment.

The cartridge or card can also include blisters containing reagent fluids for use in said system. The reagents include wash buffers, reaction buffers, and the like, and can also include an anti-drug antibody coupled to a signaling reagent (i.e. tracer). The signaling reagent can be any reagent capable of providing a signal to the optical or energy sensing means, and preferably are fluorescent dyes, radioactive reagents, phosphorescent, chemi-luminescent or other energy emitting reagents. The reader devices can thus include mechanical actuators that apply pressure for the bursting of the blisters in a controlled fashion for the delivery of the said buffers and reagents.

In other embodiments, the invention is the cartridge as described above, which can also include internal microfluidics on said substrate for carrying fluid to and from said bead sensors, as well as sample and/or fluid entry/exit port(s), together with a valve or access port, e.g., a pinch valve or elastomeric stopper for accessing said internal microfluidics.

In more detail, one embodiment of the invention disposable drug testing cartridge comprising a generally flat substrate having thereon individual bead sensors arranged in an array, wherein each bead sensor is a porous polymeric bead having a drug bound thereto, wherein said drug is two or more selected from a group containing: valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate and oxcarbazepine and biological metabolites of same.

Preferably, the drug testing cartridge has positive and negative control bead sensors and calibrator bead sensors, and every drug bead sensor is present in said array in at least duplicate or triplicate or more.

We have exemplified the invention using spherical beads, but flat bead pads may also be used, as described in US20130130933, incorporated herein by reference in its entirety for all purposes. Of course, the image analysis may change based on the shape of the bead sensor.

Usually, the drug is conjugated to said bead sensor via a linker, but this can vary depending on the bead sensor chemistry. Preferably, the bead sensor comprises crosslinked agarose, and the linker is a peptide or protein, such as BSA.

FIG. 4 shows the method of collecting oral fluid and how the results are displayed. Oral fluid sample collected by a swab or pipette (Ai) is extracted or diluted directly in assay buffer (Aii) and then delivered to the microfluidic cell (Bi) hosting bead sensors arrayed on a microchip (Bii). Step (C) shows the CCD captured images from a customized fluorescent microscope of a concentration-dependent response for a competitive-type p-BNC-based drug test (i—control 0, ii—10 ng/mL of analyte and iii—100 ng/mL of analyte). Noted are a) the decrease in signal acquired on the drug sensors (D sensor) in response to the drug, b) the absence of signal on negative control beads (−ye, CTL) and c) the consistency in signal intensity on the calibrator beads upon completion of the three independent assay runs. The schematic in C(iv) decodes the immune-components of this competitive assay approach.

The current laboratory-based iteration of this p-BNC executes a ˜10-minute test that reveals if there are AED drugs present in the body. This bead-based test functions with non-invasive oral fluid sampling, that involves use of an oral swab to brush the entire upper and lower gum line, and then insertion of the swab into a specimen collection tube to extract the sample into the assay fluid that includes the tracer antibody used in this competitive type of an immunoassay (FIG. 4). Alternatively, the oral fluid is collected by pipette and diluted directly into the assay fluid. The sample/tracer mixture is then delivered to the p-BNC flow cell equipped with a bead-array platform.

In the absence of drug in the sample, the tracer antibody specifically recognizes its corresponding drug sensor(s), producing a strong signal on the surface, as well as within the interior of the porous bead in the array. In the presence of drug in the sample the binding of the tracer is reduced in a drug specific, dose dependent manner. The array allows for 3-4 bead redundancy of bead sensors per drug target, which translates into higher accuracy and more precise measurements. As seen in the experiments, the BNC array has beads coated with BSA-drug conjugate, negative controls beads coated with BSA alone, and with calibrator beads used as internal controls.

### Experiment 1

Currently two elements are needed to measure the AED content in oral fluid. First is the lab-based imaging station with a fluidic control system (seen in FIG. 1A). This was utilized for all experiments discussed. However, a portable imaging system could also be used and is under development. Further, there are also portable systems that are commercially available. The second element is the packaged microfluidic sample processing and immune-analysis BNC. This is the functional component for the detection and quantitation of the targeted drug and/or its metabolites.

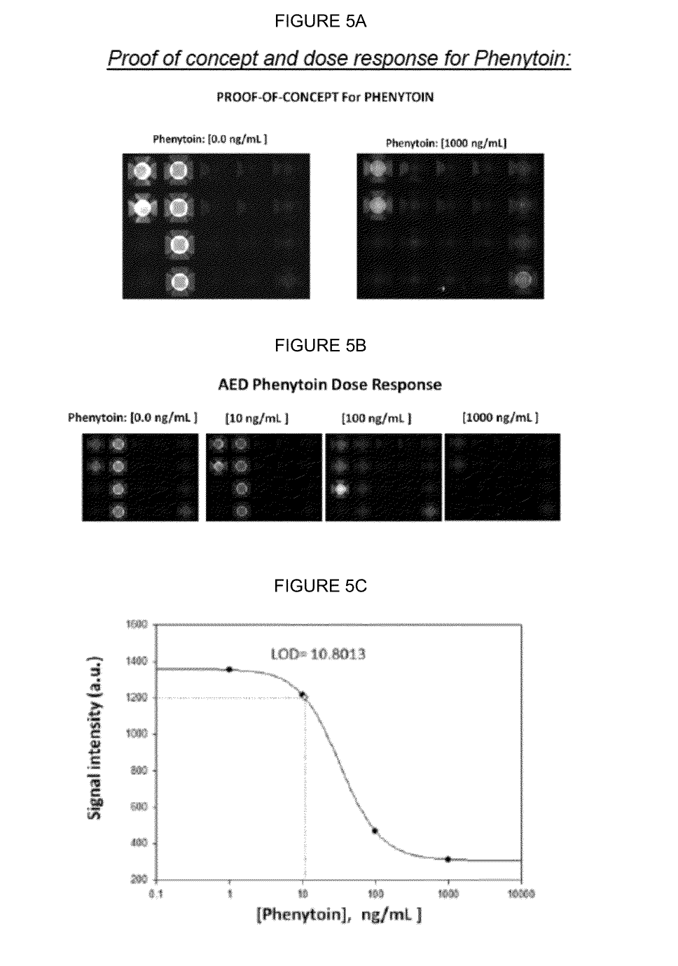

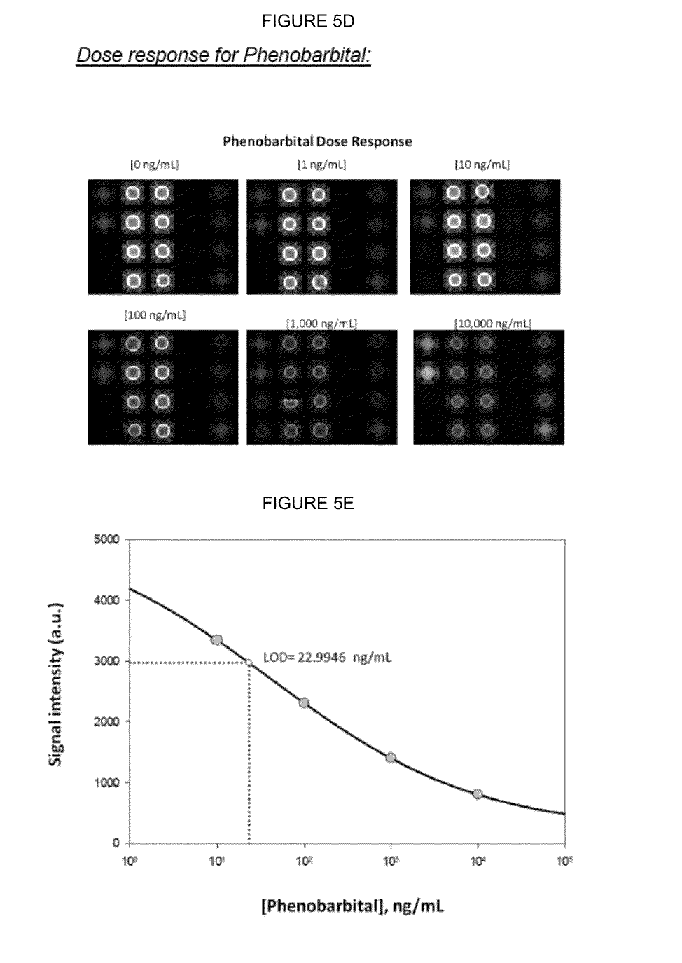

With these two elements, proof of concept experiments were conducted for phenytoin and phenobarbital. The AED test chips were developed and calibrated across a range of concentrations for these two target drugs. FIG. 5A is a proof of concept for 0 ng/ml and 1000 ng/ml phenytoin. FIG. 5B shows images of a proof of concept sequence of assay runs targeting 0, 10, 100 and 1000 ng/mL of a drug standard for phenytoin that ultimately allows for the generation of a dose response (calibration) curve, as shown in FIG. 5C, used to interpolate the concentration of the drug in oral fluid samples. FIG. 5D shows a dose response for phenobarbital for 0, 1, 10, 100, 1,000 and 10,000 ng/ml. FIG. 5E shows the dose response curve.

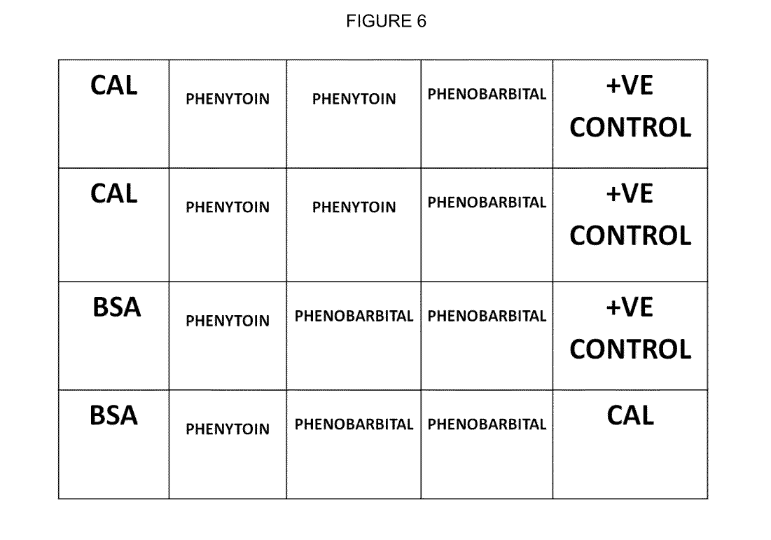

The bead-based BNC competitive type of immunoassay was executed on the lab-on-a-chip (LOC) system as follows: The tracer antibody was mixed with the saliva sample and the mixture delivered over a period of about 5-10 minutes (7.5 minutes) to the array of beads in the microfluidic cell. The array had beads coated with BSA-drug conjugate, negative controls beads coated with BSA alone, and calibrator beads used as internal controls. FIG. 6 displays the location of these beads within the BNC used.

In the absence of drug in the sample, the tracer antibody efficiently recognized and bound to the bead sensors for which it was specific (i.e. bead coated with the drug of interest) and thus produced a fluorescent signal within and around the bead. In the presence of drug in the sample, which competed with the drug on the bead for binding to the tracer, the tracer-derived signal on the drug-sensitized bead was reduced in a dose dependent manner.

FIG. 5A shows the results for phenytoin. As expected, the brightest signal was for the calibration beads and the sample beads for a phenytoin-free sample. Upon introduction of a phenytoin-containing sample, the fluorescent signal decreased in intensity to barely being visible at a concentration of 1000 ng/mL, as shown in FIG. 5B. From the plot of concentration and signal intensity in FIG. 5C, the limit of detection (LOD) was approximately 10.8 ng/mL.

FIG. 5D-E shows the results for phenobarbital. Here, the brightest signals were for the phenytoin sample beads and the phenobarbital sample beads for a phenobarbital-free sample. Again, the fluorescence signal decreased in intensity as the concentration of phenobarbital increased. The concentration and signal intensity plot in FIG. 5E shows that phenobarbital had a high limit of detection at approximately 22.99 ng/mL.

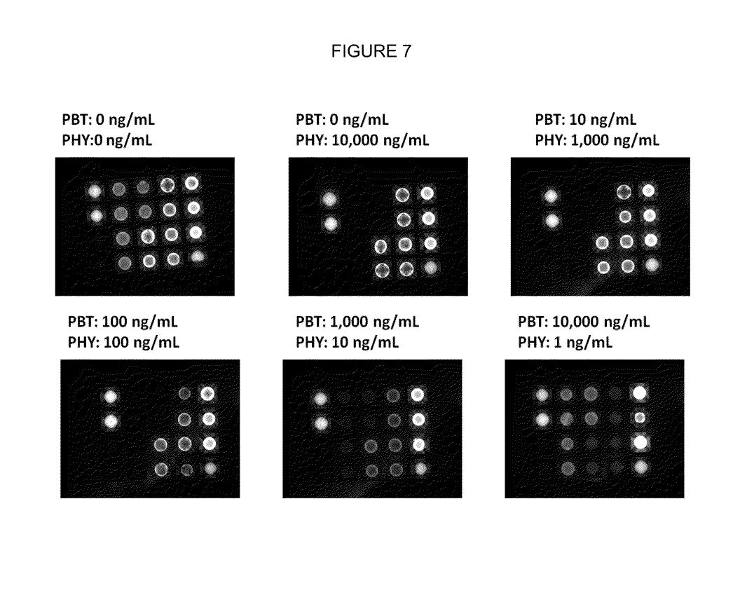

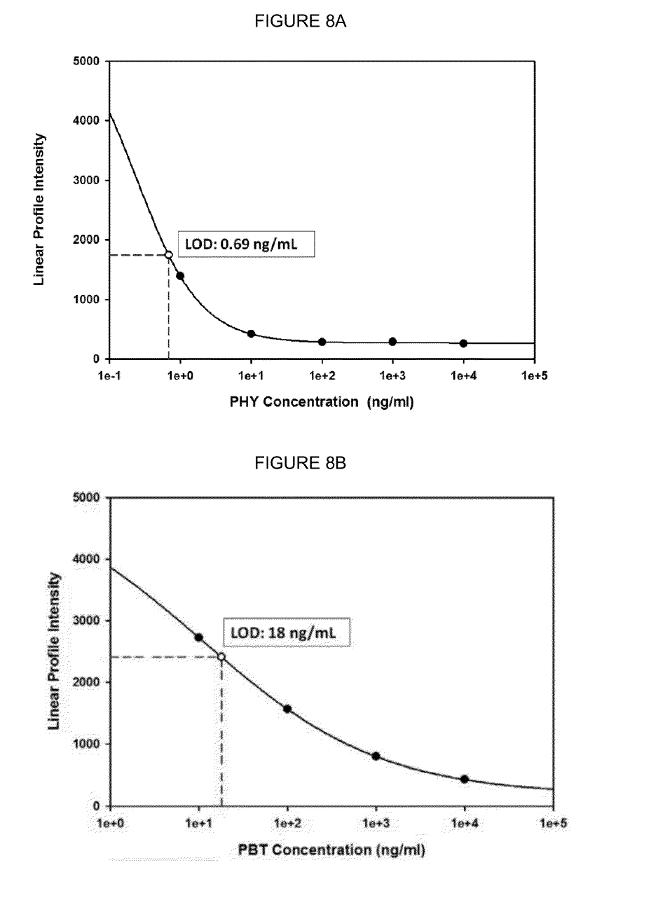

FIG. 7 displays raw images of the BNC chips during the assay for a variety of control samples with known concentrations of phenobarbital and phenytoin. As the concentration of PHY decreases, the beads in column 2 begin to appear around 10 ng of phenytoin. As the concentration of PBT increases, the beads in column 3 and 4 begin to decrease in signal intensity. Based on these experiments, a plot of the concentration versus the linear profile intensity are shown in FIGS. 8A-B. In FIG. 8A, it is indicated that the limit of detection based on this parameter was about 0.69 ng/mL for PHY, and in FIG. 8B the limit of detection was about 18 ng/mL for PBT. The large difference in LOD for both analytes was maintained.

Proof of concept was considered met by the above experiments because the BNC provided high signal to noise result, whereby the analyte-specific beads provided a significantly higher signal than the control beads in response to the tracer and the BNC demonstrated efficient competition between drug analyte and tracer, whereby the signal on drug-sensitized beads was significantly reduced in the presence of the specific drug.

### Experiment 2

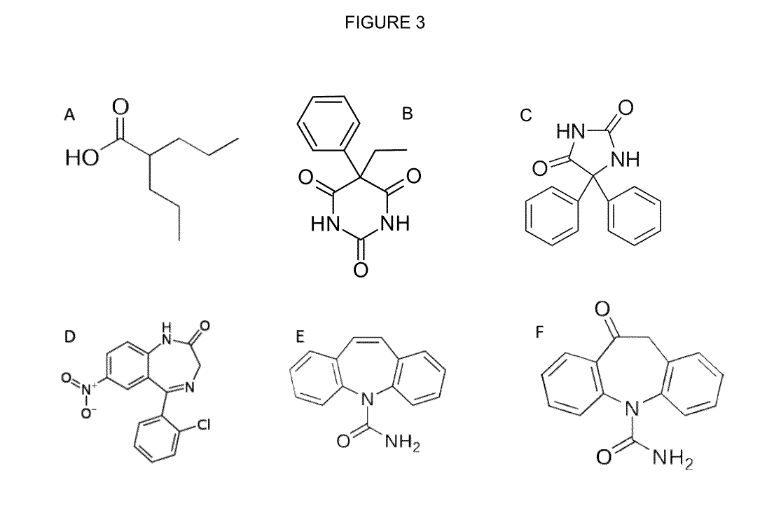

The presently disclosed multiplexed BNC assay chip was tested on a pilot population of volunteer epilepsy patients seen at the neurology practice of UT Physicians at the Texas Medical Center to determine the concentration of phenytoin (FIG. 3B) and phenobarbital (FIG. 3C) in these patients.

As with most assay development, the success of the test relies heavily on the availability of high quality reagents. While most AEDs can be analyzed, only two commonly used AEDs were tested for the trial run because a plethora of commercial reagents exist for their assay, and these could be used as external controls.

The patients were on a PBT and/or PHY treatment regime. A serum sample and multiple saliva samples were obtained at each visit for each patient. Oral fluid samples were collected by swabbing the entire upper and lower gum line with an oral swab which was then diluted by inserting the swab into a specimen collection tube containing Aware Messenger transfer matrix (buffered sample) or by collecting passive drool (clear sample) The buffered sample was immediately frozen at −80° C. for analysis.

The serum sample (also called the ‘gold standard’) was processed by particle enhanced turbidimetric immunoassay (PETINIA) in a clinical chemistry laboratory. A control group of salivary samples was suspended in a phosphate buffer and assayed by gas chromatography-mass spectrometry (GC/MS) at a commercial toxicology laboratory.

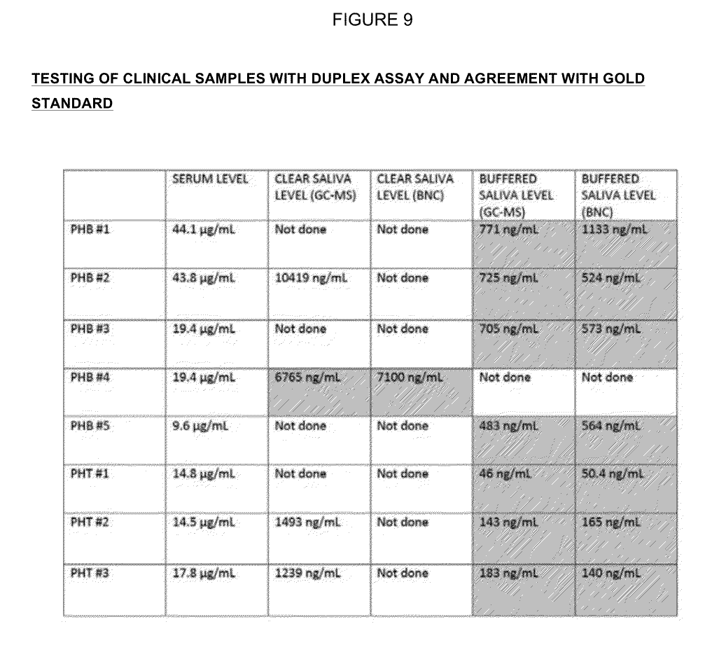

The saliva samples were run using sequentially diluted competitive mix against 3 clinical samples of PHT and 5 of PHB for a total of 8 samples. The results of this study are shown in FIG. 9.

As can be seen in FIG. 9, the BNC easily detected the PHB or PHT in the buffered saliva. The estimated concentrations were favorable comparable to those seen in the GC/MS analysis. As such, the BNC provided reliable detection with high sensitivity.

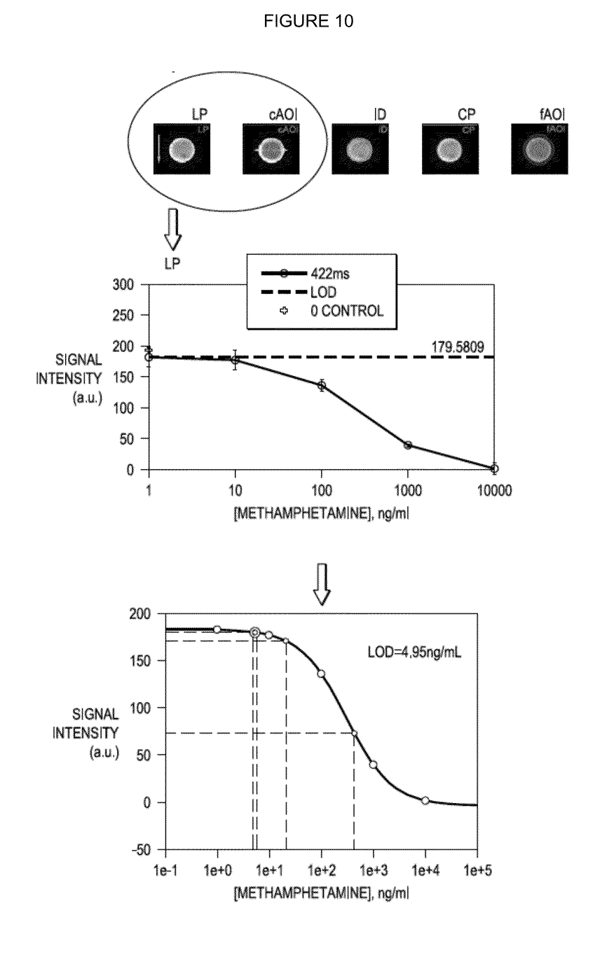

The assays benefited from automated image and data analysis macros developed specifically for this application. Five dedicated image analysis “probing” strategies are shown in FIG. 10, including Line profile (LP), circular area of interest (cAOI), integrated density (ID), circular profile (CP) and fixed AOI.

The algorithm compiled results for each bead, statistical analysis with exclusion of outliers within each group of beads and output log files with the average, standard deviation and coefficient of variance for each group that can be inserted and further processed into a Microsoft Excel environment. Intensity versus concentration calibration curves were constructed with best-fit regression analysis for determination of unknown sample concentration. Data obtained from the testing of drug standards and zero antigen controls were then entered and processed to derive the dose response curves, as well as assay characteristics such as limit of detection, assay range and precision.

The dose response data as well as data obtained from the testing of samples were entered into unknown prediction equations according to standard curves obtained for each analyte on the system to determine the drug concentrations. Further enhancement in data quality was obtained by using image acquisition with various exposure times. The latter feature was developed with the flexibility that allows selective independent analysis for each assay using the optimal integration time for each target drug under the various conditions tested.

Line Profile (LP) and circular Area of Interest (cAOI) were the two image analysis methods that consistently provided the best results. Hence, these two methods were selected and used extensively for the validation of the drug tests with respect to assay performance studies.

For the Line Profile, a series of lines going through about 80% of the beads were profiled for the maximum intensities (or maxima). Because the signal is typically lower at the center of the beads, the product of a line profile is typically two maxima at the edge of the bead. All measurements were averaged and outliers identified and removed according to well established non-proprietary outlier removal routines (median, Grubb's, or Dixon tests).

For circular Area of Interest, a series of concentric areas centered on the center of the beads, and starting with a diameter of only a few pixels are drawn with increasing radii. For each of these circular areas, the average intensity per pixel was calculated and the circle was increased until it has exceeded the size of the bead by 10%. The maximum signal obtained typically at the bead periphery can be determined from the highest circular area value.

Each of the following reference is incorporated by reference herein in its entirely.

61/498,761, US20120322682, WO2012154306, WO2012065117, WO2012065025, WO2012021714, WO2007134189, WO2012065025 , 61/815,305 filed Apr. 24, 2013.

Baumann RJ. Salivary monitoring of antiepileptic drugs. J. Pharm. Pract. 2007; 20:147-157.

Greenaway C, Ratnaraj N, Sander J W, Patsalos P N. Saliva and serum lacosamide concentrations in patients with epilepsy. Epilepsia 2011; 52:258-263.

Miles M V, Tang P H, Ryan M A et al. Feasibility and limitations of oxcarbazepine monitoring using salivary monohydroxycarbamazepine (MHD). Ther. Drug Monit. 2004; 26:300-304.

Miles M V, Tang P H, Glauser T A et al. Topiramate concentration in saliva: an alternative to serum monitoring. Pediatr. Neurol. 2003; 29:143-147.

al-Za'abi M., Deleu D, Batchelor C. Salivary free concentrations of anti-epileptic drugs: an evaluation in a routine clinical setting. Acta Neurol. Belg. 2003; 103:19-23.

Suzuki Y, Uematsu T, Mizuno A, Ninchoji T, Fujii K, Nakashima M. Analysis of the transport of valproic acid into saliva from serum. Biol. Pharm. Bull. 1994; 17:340-344.

Umstead G S, McKernan T M. Salivary phenytoin concentrations in geriatric patients. Clin. Pharm. 1982; 1:54-58.

Troupin A S, Friel P. Anticonvulsant level in saliva, serum, and cerebrospinal fluid. Epilepsia 1975; 16:223-227.

Gorodischer R, Burtin P, Verjee Z, Hwang P, Koren G. Is saliva suitable for therapeutic monitoring of anticonvulsants in children: an evaluation in the routine clinical setting. Ther. Drug. Monit. 1997; 19:637-642.

Reynolds F, Knott C. Saliva monitoring of anticonvulsants. J Clin Chem Clin Biochem. 1989; 27:226-227.

Danhof M, Breimer D D. Therapeutic drug monitoring in saliva. Clin Pharmacokinet 1978; 3:39-57.

Horning M G, Brown L, Nowlin J, Lertratanangkoon K, Kellaway P, Zion T E. Use of saliva in therapeutic drug monitoring. Clin. Chem. 1977; 23:157-164.

The following patents or patent applications by the McDevitt group are also incorporated by reference in their entireties for all purposes:

U.S. Pat. No. 8,257,967.

US20060073585, US20060079000, US2006234209.

U.S. Pat. No. 7,781,226, U.S. Pat. No. 8,101,431, U.S. Pat. No. 8,105,849, US20060257854, US20060257941, US20060257991.

U.S. Pat. No. 8,377,398.

US20100291431.

US20080050830.

US20080038738.

US20080176253.

US20080300798.

US20120208715.

US20130130933.

US20132095580.

US20130274136.

US20140094391.

US20120322682.

US20140235487.

61/841,090 and Ser. No. 14/319,497, filed Jun. 28, 2013 and Jun. 30, 2014.

61/833,750 and Ser. No. 14/299,908, filed Jun. 11, 2013 and Jun. 9, 2014.

61/815,305 and Ser. No. 14/258,770, filed Apr. 24, 2013 and Apr. 22, 2014.

What is claimed is:

1. A disposable drug testing cartridge comprising a generally flat substrate having thereon individual bead sensors arranged in an array, wherein each said bead sensor is a porous polymeric bead having a drug bound thereto to form a drug bead sensor, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, oxcarbazepine, and biological metabolites or derivatives of same.

2. The disposable drug testing cartridge of claim 1, further comprising internal microfluidics on said substrate for carrying fluid to and from said bead sensors.

3. The disposable drug testing cartridge of claim 2, further comprising a sample entry port.

4. The disposable drug testing cartridge of claim 3, further comprising at least one reagent blister fluidly connected to said bead sensors.

5. The disposable drug testing cartridge of claim 4, further comprising at least one waste fluid chamber fluidly connected to and downstream of said bead sensors.

6. The disposable drug testing cartridge of claim 4, further comprising positive and negative control bead sensors and calibrator bead sensors.

7. The disposable drug testing cartridge of claim 4, wherein every said drug bead sensor is present in said array in at least duplicate.

8. The disposable drug testing cartridge of claim 4, wherein every said drug bead sensor is present in said array in at least triplicate.

9. The disposable drug testing cartridge of claim 4, wherein said drug is conjugated to said bead sensor via a linker.

10. The disposable drug testing cartridge of claim 1, said cartridge further comprising:
a) one or more reagent chambers fluidly connected to and upstream of said array; and
b) one or more waste fluid chambers fluidly connected to and downstream of said array;
c) a sample inlet upstream and fluidly connected to said one or more reagent chambers; and
d) wherein each bead sensor is a porous polymeric bead of size between 50-300 nm ±10%.

11. An assay for the monitoring of anti-epilepsy drug concentration in saliva, said assay comprising:
a) obtaining a sample of oral fluid from a patient;
b) immunologically testing said sample to determine the level of anti-epileptic drugs; and
c) wherein said testing is conducted on an array of agarose beads, conjugated to anti-epileptic drugs, and wherein signal from said array of agarose beads is analyzed by circular area of interest or line profiling or both.

12. The assay of claim 11, wherein said anti-epileptic drugs are selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites of same.

13. An anti-epileptic drug testing assay system comprising:
a) a microfluidic lab-on-chip based reverse competitive immunoassay that comprises a disposable cartridge and a separate reader, wherein said cartridge fits into a slot on said reader, and said reader performs said competitive immunoassay and outputs a result;
b) said cartridge comprising:
i) a generally flat substrate having embedded microfluidic channels connecting an inlet port to an embedded downstream assay chamber having a transparent cover and containing a removable array of bead sensors;
ii) one or more reagent chambers fluidly connected to and upstream of said assay chamber; and
iii) one or more waste fluid chambers fluidly connected to and downstream of said assay chamber;
iv) wherein each bead sensor is a porous polymeric bead of size between 50-300 nm ±10% having a drug conjugated thereto, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites or derivatives of same; and
c) wherein said reverse competitive immunoassay has a lower limit of detection for each of said drugs of <50 ng/ml and a detection range of at least four orders of magnitude.

14. The drug testing assay of claim 13, wherein said cartridge comprising 4 or more of said drugs.

15. The drug testing assay of claim 13, wherein said cartridge comprising each of said drugs.

16. A kit, comprising the cartridge of claim 13 wrapped in an airtight package, a vial of assay fluid, and an oral swab.

17. A anti-epileptic drug home testing assay kit comprising:
a) a microfluidic lab-on-chip based reverse competitive immunoassay that comprises a disposable cartridge;
b) a sample collection and solubilization device comprising:
i) a container closed with a removable cap;
ii) a lower portion of said container having an assay fluid separated from an upper portion of said container by a piercable membrane;
iii) said cap comprising a flexible bulb passing through said cap and fluidly connected to a hollow stem ending in a point, a lower portion of said stem being coated with an absorbent or bristled material for collecting a biological sample;
iv) wherein said device is proportioned to store said cap with said stem and said swab in said upper portion of said container, but can reach said buffer when said point pierces said piercable membrane, and said flexible bulb can be used to draw up and deliver said buffer;
c) said cartridge comprising:
i) a generally flat substrate having embedded microfluidic channels connecting an inlet port to an embedded downstream assay chamber having a transparent cover and containing a removable array of bead sensors;
ii) one or more reagent chambers fluidly connected to and upstream of said assay chamber;
iii) one or more waste fluid chambers fluidly connected to and downstream of said assay chamber; and
iv) wherein each bead sensor is a porous polymeric bead of size between 50-300 nm ±10% having a drug conjugated thereto, wherein said drug is selected from three or more of valproic acid, phenobarbital, phenytoin, clonazepam, carbamazepine, ethosuximide, felbamate, tiagabine, levetiracetam, lamotrigine, pregabalin, gabapentin, topomax, zonisamide, perampanel, lacosamide, topiramate, and oxcarbazepine and biological metabolites or derivatives of same.
